(19) United States
(12) Patent Application Publication
(10) Pub. No.: US 2021/0138227 A1
(43) Pub. Date: May 13, 2021

(54) CARDIAC ASSIST DEVICE WITH INTEGRALLY TEXTURED MEMBRANE

(71) Applicant: Cardiac Assist Holdings, LLC, Ann Arbor, MI (US)

(72) Inventors: Kurt A. Dasse, Wilmington, DE (US); Barry Gelman, Wilmington, DE (US); Allen B. Kantrowitz, Wilmington, DE (US)

(73) Assignee: Cardiac Assist Holdings, LLC, Ann Arbor, MI (US)

(21) Appl. No.: 16/492,539

(22) PCT Filed: Mar. 8, 2018
(86) PCT No.: PCT/US2018/021458

§ 371 Date: Sep. 9, 2019

Related U.S. Application Data
(60) Provisional application No. 62/468,825, filed on Mar. 8, 2017.

(51) Int. Cl. A61M 60/139; A61M 60/268; A61M 60/497; A61M 60/117

(52) U.S. Cl. CPC A61M 60/139; A61M 60/268; A61M 2205/04; A61M 60/117; A61M 2205/0216; A61M 60/497

(57) ABSTRACT

A cardiac pump and an assist system is provided to increase blood ejection from a compromised heart. An implantable cardiac pump acting as an assist device includes an attachment system and locating features that enable a minimally invasive procedure to implant and deploy one or more aortic blood pumps in a patient. The cardiac pumps are replaceable without resort to a surgical procedure. Monitoring of cardiac pump operation allows for replacement in advance of chamber failure. The cardiac pump and assist system do not appreciably sheer blood being accelerated through inflation-deflation cycling so as to limit clot associated side effects of operation of a cardiac assist device.

### RELATED APPLICATIONS

This application claims priority benefit of U.S. Provisional Application Ser. No. 62/468,825 filed 8 Mar. 2017; the contents of which are hereby incorporated by reference.

### FIELD OF THE INVENTION

The present invention in general relates to medical devices and systems, and more particularly to a minimally invasive cardiac assist device and method of implantation thereof.

### BACKGROUND OF THE INVENTION

Heart disease is one of the leading causes of death. Currently, medical science cannot reverse the damage done to the cardiac muscle by heart disease. The only known solution is a heart transplant. However, the number of cardiac patients in need of a heart transplant far exceeds the limited supply of donor hearts available.

The scarcity of human hearts available for transplant, as well as the logistics necessary to undertake heart transplant surgery, makes a permanently implantable cardiac assist device the only viable option for many heart patients. An aortic blood pump can be permanently surgically implanted in the wall of the aorta to augment the pumping action of the heart. The aortic blood pump is sometimes referred to as a mechanical auxiliary ventricle assist device, dynamic aortic patch, or permanent balloon pump. Alternatively, the aortic blood pump can be inserted endovascularly.

Typically, the aortic blood pump includes a flexible bladder to be inflated and deflated in a predetermined synchronous pattern with respect to the diastole and systole of the patient to elevate aortic blood pressure immediately after aortic valve closure. Inflation and deflation of the bladder can be accomplished by means of a supply tube connected to the bladder and can be connected to a percutaneous access device (PAD). The PAD can be permanently surgically implanted in a patient's body to provide a through-the-skin coupling for connecting the supply tube to an extra-corporeal fluid pressure source. Alternatively, the fluid pressure source can be implanted wholly within the body, energized by an electromagnetic means across intact skin, or energized by chemical energy found within the body or some other means. Electrical leads from electrodes implanted in the myocardium are likewise brought out through the skin by means of the PAD. The aortic valve status or any cardiovascular parameter that is associated with this status can be employed to control the fluid pressure source to inflate and deflate the inflatable chamber in a predetermined synchronous relationship with the heart action.

The aortic blood pump acts to assist or augment the function of the left ventricle and is typically restricted to use in patients who have some functioning myocardium. The aortic blood pump does not need to be operated full time, and in fact, can be operated periodically on a scheduled on-time, off-time regimen. Typically, the patient can be at least temporarily independent of the device for periods of one to four hours or more, since the intra-aortic blood pump does not require continuous operation.

U.S. Pat. No. 4,051,840 discloses a dynamic aortic patch that is surgically implanted in the thoracic aorta and is systematically inflated and deflated to generate pressure waves in the bloodstream. The pressure waves assist the heart by augmenting the circulation of the blood through the body. The patch includes a flexible inflatable bladder and an independent envelope. The envelope has a reinforced surface for limiting and directing inflation of the bladder inwardly toward the lumen of the aorta.

U.S. Pat. No. 6,471,633 discloses a dynamic aortic patch with an elongate bladder having a semi-rigid shell body portion and a relatively thin membrane portion defining an inflatable chamber. At least one passage extends through the shell body defining an opening in the inner surface of the shell body. The flexible membrane can be continuously bonded to the shell body adjacent the peripheral side edge to define the enclosed inflatable chamber in communication with the passage. The membrane has a reduced waist portion defining a membrane tension zone adjacent to the opening of the passage into the chamber to prevent occluding the entrance while deflating the chamber. An outer layer can be bonded to the outer side of the semi-rigid wall portion of the aortic blood pump and cut with a freely projecting peripheral edge portion to provide a suture flange for suturing the aortic blood pump in place within an incision in the aorta.

Further details regarding the structure and function of the aortic blood pump and associated devices and controls can be obtained from U.S. Pat. No. 6,511,412 issued Jan. 28, 2003; U.S. Pat. No. 6,471,633 issued Oct. 29, 2002; U.S. Pat. No. 6,132,363 issued Oct. 12, 2000; U.S. Pat. No. 5,904,666 issued May 18, 1999; U.S. Pat. No. 5,833,655 issued Nov. 11, 1998; U.S. Pat. No. 5,833,619 issued Nov. 10, 1998; U.S. Pat. No. 5,242,415 issued Sep. 7, 1993; U.S. Pat. No. 4,634,422 issued Jan. 6, 1987; and U.S. Pat. No. 4,630,597 issued Dec. 23, 1986 which are incorporated by reference in their entirety herein.

While conventional aortic balloon pumps are well known to the art, driveline infection remains one of the most frequent and costly adverse events associated with cardiac assist devices at the percutaneous access device (PAD), as well as systemic infections due to ascending microbial invasion.

Ventricular Assist Device (LVAD) driveline infections (DLI) are the most common type of infection associated with implantable pumps. These infections occur at the skin penetration site because current devices require an external power source with energy supplied via a tunneled percutaneous driveline. Driveline infections frequently occur because the driveline exit site creates a conduit for entry of bacteria. DLI, along with gastrointestinal bleeding (GIB) and stroke, are the leading causes of unplanned readmission for patients with an LVAD

Furthermore, while there have been many advances in heart assist devices there is a significant need to minimize the risk of thromboembolic complications and exit site infections. In addition, a stable aortic blood pump implant is desirable, since the constant movement of blood, movement of the vessel wall and the movement of the pump itself can result in deformation of the pump and vessel damage at blood/pump and vessel/pump interface areas.

There is a continuing need for a cardiac pump including a structure adapted to maintain implant stability that is implanted with minimally invasive surgical incisions with accurate location placement that significantly minimizes the risk of thromboembolic complications and exit site infections

### SUMMARY

A cardiac assist device includes an inflatable cardiac pumping chamber with an integrally textured polymeric membrane contacting blood upon insertion in a subject aorta. A drive line is in fluid communication with the inflatable cardiac pumping chamber. An external drive unit or fluid supply is in fluid communication with the drive line.

An inflatable cardiac pumping chamber is provided having a membrane moving to change a volume of the chamber based on fluid input from an inflation source. A drive line is in fluid communication with the inflatable cardiac pumping chamber and the inflation source, wherein the improvement lies in: the membrane being an integrally textured polymeric membrane contacting blood upon insertion in a subject aorta.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which like reference numerals refer to like parts throughout the several views, and wherein:

FIGS. 1A-1N are a series of partial cutaway perspective views showing the implantation and deployment of a cardiac assist device in accordance with embodiments of the invention;

FIGS. 2A-2I are a series of simplified cross-sectional views further describing the implantation and deployment of a cardiac assist device of FIGS. 1A-1N in accordance with embodiments of the invention;

FIGS. 3A-3I are a series of cross-sectional views of a needle and plunger to assist in the implantation and deployment of a cardiac assist device in accordance with embodiments of the invention;

FIGS. 4A-4C are a series of perspective cross-sectional views of a needle and plunger to assist in the implantation and deployment of a cardiac assist device in accordance with embodiments of the invention;

FIG. 5A is a cross-sectional side view of a percutaneous access device implanted in a patient for providing a power or actuating connection to a cardiac assist device according to an embodiment of the invention;

FIG. 5B illustrates the use of an implanted transcutaneous energy transfer module (TET) for providing power or actuating connection to a cardiac assist device according to an embodiment of the invention;

FIG. 5C illustrates a cross-sectional side view of a single percutaneous access portal implanted in a patient for providing a power or actuating connection to two cardiac pumps according to an embodiment of the invention;

FIG. 5D illustrates an external power source or pump connected via a percutaneous access device to a cardiac assist device according to an embodiment of the invention;

FIG. 5E illustrates multiple ventricular assist devices in a patient aorta in accordance with embodiments of the invention;

FIGS. 6A-6F are a series of cross-sectional side views showing the implementation and actuation of a ventricular assist device in accordance with embodiments of the invention;

FIGS. 7A-7D are a series of partial cutaway perspective views showing the implantation and deployment of a cardiac assist device in accordance with embodiments of the invention;

FIGS. 8A and 8B are cross-sectional views of an aortic assist device as a flexible encasement with a balloon inside in a deflated and inflated state, respectively, in accordance with embodiments of the invention;

FIG. 9 illustrates the integrally textured polyurethane membrane blood interface on the surface of the cardiac assist device in accordance with embodiments of the invention;

FIG. 10 illustrates the wearable and implanted components of the cardiac assist system in accordance with embodiments of the invention;

FIG. 11 illustrates the cardiac assist device, aortic access device, and PAD of an embodiment of the cardiac assist system;

FIG. 12A-12C are close-up views of a textured pump in an aorta, an aortic access device, and a PAD, respectively in accordance with embodiments of the invention;

FIG. 13A illustrates an axillary graft for entry into the aorta; and

FIG. 13B illustrates entry into the aorta via an aortic access port.

### DETAILED DESCRIPTION OF THE INVENTION

A cardiac pump and an assist system according to the present invention have utility to increase blood ejection from a compromised heart. An implantable cardiac pump acting as an assist device provided by the present invention includes an attachment system and locating features that enable a minimally invasive procedure to implant and deploy one or more aortic blood pumps in a patient. Embodiments of the insertable cardiac pump are replaceable without resort to a surgical procedure. Still other embodiments of the present invention allow for monitoring of cardiac pump operation to allow for replacement in advance of chamber failure. Additionally, it has been discovered that in contrast to existing cardiac assist devices that cause sheering of blood as evidenced by conformation changes in the von Willebrand factor found in blood, embodiments of an inventive cardiac device do not appreciably sheer blood being accelerated through inflation-deflation cycling so as to limit clot associated side effects of operation of a cardiac assist device.

Embodiments the inventive cardiac assist system (CAS) may be implanted using a minimally-invasive surgical (MIS) technique for use in an acute hospital setting and for longer-term chronic applications outside of the hospital setting. Embodiments of the inventive cardiac assist system address major limitations of current designs by incorporating advances in four key components: a wearable external drive unit (EDU) 90, a percutaneous access device (PAD) 92, an aortic access device (AAD) 94, and an integrally textured membrane aortic pump (TAP) (MIS counterpulsation cardiac assist device) 96 as shown in FIG. 11. FIG. 10 further illustrates the wearable and implanted components of the cardiac assist system, where an external drive unit (EDU) 90, skin connector PAD 92′, battery pack 98, and a cardiac assist device 96 are shown. A representative conventional system absent a textured is detailed in U.S. Pat. No. 6,735,532. Furthermore, embodiments of the inventive CAS are designed to be safely turned on and off at will in contrast to currently available continuous-flow devices, which must always remain on. A conventional PAD operative herein is detailed in U.S. Pat. No. 5,833,655 or 8,383,407.

In a specific embodiment the external drive unit is pneumatic with a driveline (DL) incorporating polymeric velour at the skin access site in addition to the TAP. The velour in some inventive embodiments being polyester. Embodiments of the integrally textured pump membrane (see FIG. 9) have been found to minimize thromboembolic complications, neurologic dysfunction and bleeding. M. J. Menconi et al., J. of Cellular Biochem., 57:557-573 (1995). In a specific inventive embodiment, the textured blood-contacting surface is based on integrally textured polymer (ITP) formed as a membrane and in an exemplary embodiment an integrally textured (IT) polyurethane. Other suitable materials for an ITP illustratively include polyamides, polyimides, polyesters, polycarbonates, copolycarbonate esters, polyethers, polyetherketones, polyetherimides, polyethersulfones, polysulfones, polyvinylidene fluoride, polybenzimidazoles, polybenzoxazoles, polyacrylonitrile, cellulosic derivatives, polyazoaromaties, poly(2,6-dimethylphenylene oxide), polyphenylene oxides, polyureas, polyurethanes, polyhydrazides, polyazomethines, polyacetals, cellulose acetates, cellulose nitrate, ethyl cellulose, styrene-acrylonitrile copolymers, brominated poly(xylylene oxide), sulfonated poly(xylylene oxide), tetrahalogen-substituted polycarbonates, tetrahalogen-substituted polyesters, tetrahalogen-substituted polycarbonate esters, polyquinoxaline, polyamideimides, polyamide esters, polysiloxanes, polyacetylenes, polyphosphazenes, polyethylenes, polyphenylenes, poly(4-methylpentene), poly(trimethylsilylpropyne), poly(trialkylsilylacetylenes), polyureas, polyurethanes, blends thereof, block copolymers thereof; a fiber or particle filled forms of any of the aforementioned.

The properties of a fold-free ITP formed of polyurethane for use in a blood contacting surface are detailed in M. J. Menconi et al., J. of Cellular Biochem., 57:557-573 (1995). In a specific embodiment, the integrally textured membrane has pleats. The pleats are present in FIG. 9, but not shown for visual clarity. The pleats used in the present invention, are parallel and longitudinal; spiral, or a combination thereof. The purpose of pleats is to avoid creasing which is the failure mechanism by spreading the expansion forces over a larger area. Not intending to be limited to a specific theory, the ITP is used to achieve at least one of the following objectives of: promote natural growth of a biologic lining on the surface of the in-dwelling pump to reduce the need for anticoagulation and the risk of thromboembolic events; promote washing of the surface to minimize stasis and thrombus formation; minimize strain on the ITP; minimize elongation radially and longitudinally to avoid fatigue of the ITP; minimize stretching and stress distribution along a balloon embodiment; promote a sweeping effect through the channels in the non-expanded state to wash the surface; or a combination thereof.

Thus, when the integrally textured polyurethane is exposed to the blood, the integrally textured membrane, such as one formed of polyurethane, is believed to develop a biofilm that in turn has puripotent cells attach thereto. These cells then flatten and take on the appearance and function of epithelial cells.

Balloon implantable pumps and cardiac assist devices stitched to a subject aorta are taught in application Ser. No. 13/971,852; U.S. Pat. No. 8,540,618; or U.S. Pat. No. 6,471,633 that are adapted to have an inventive integrally textured coated surface as detailed herein.

Furthermore, the PAD used in inventive embodiments of the cardiac assist system promotes the formation of a natural biologic seal between the skin and the device to form a barrier to microbial invasion into the body. Embodiments of the PAD may also illustratively be used for other devices including peritoneal dialysis catheters and chronic indwelling venous access catheters that require skin penetration.

Embodiments of the inventive implantable pump may be inserted using a well-established minimally invasive surgical (MIS) procedures, illustratively including insertion by creating a side-arm, axillary access port for introduction of the pump directly or via a standard Seldinger Technique. In a specific embodiment a wearable hydraulic EDU may be used to drive the textured aortic pump, where the EDU is of reduced size, weight, and noise. The reduced size, weight, and noise of the EDU is more patient-friendly and improves the quality of life of the patient and facilitates the ability of the patient to ambulate and exercise.

In a specific inventive embodiment an aortic access device (V-Port) is designed to facilitate MIS surgical insertion of the textured aortic pump or any other device that requires access into the body through the aorta. This aortic access device is connected to the PAD at the skin level and attached to the aorta on the distal end.

The CAS devices function by inflating an actuator at the onset of diastole to increase aortic pressure during ventricular relaxation, and to deflate during systole, reducing left ventricular afterload. The effect is to delay the arterial pressure peak so that it occurs during diastole, a period of decreased peripheral resistance. This improves circulation while minimizing the energy requirement of the weakened left ventricle.

A process of operating a cardiac assist device includes cyclically inflating and deflating one or more inflatable cardiac pumping chambers with timing and parameters as to pressure, deflection and speed of inflation to increasing cardiac output of the patient.

Embodiments of the inventive cardiac assist system (CAS) offer the following advantages:

Interruptibility. Circulatory assistance provided by the counter-pulsating CAS device can be modulated at will, based on patient need. The CAS can be stopped and restarted for short periods as needed without risk of catastrophic failure. Wean patient by volume.

Minimal need for anticoagulation. The blood-biomaterial interface is rapidly covered with native tissue, minimizing the risk of thrombus formation. In addition, the counter-pulsation timing of the CAS devices has been designed to produce sheer stress like that of normal ventricular function. These innovations minimize the need for anticoagulants.

Reduced infection rate. Embodiments of the CAS devices include a percutaneous access device pre-coated with the recipient's dermal fibroblasts. These dermal fibroblasts inhibit epidermal down growth, preventing sinus tract formation along the driveline; an environment that supports microbial growth.

In addition to minimizing driveline infections, the CAS aims to reduce thromboembolic complications, bleeding and neurologic dysfunction.

Minimally invasive surgical implantation. CAS devices may be implanted using a minimally invasive surgical (MIS) approach through the V-Port as a Vascular Access Port. This will minimize surgical complications and allow implantation by specialists trained in MIS techniques.

Reduced hospitalization costs. Minimally invasive implantation aims to shorten operating room time, recovery time and length of stay. Reduced anticoagulant use, which lowers associated adverse events, also results in an overall reduction in medical costs.

An embodiment of a system 10 for the attachment and deployment of a cardiac pump is described in FIGS. 1A-1N as a series of partial cutaway perspective views in conjunction with the cross-sectional views of FIGS. 2A-2I that further describe the implantation and deployment of a cardiac assist device inclusive of the pump depicted with respect to FIGS. 1A-1N. In FIG. 1A an endo-aortic securement 12 (hereinafter referred to as securement 12) connected to a non-distensible collapsed sub-endothelial pocket 20 (hereinafter referred to as pocket 20 or synonymously as a secondary luminal confinement) are shown implanted in a patient vessel V, illustratively including the aorta. Implantation of the endo-aortic securement 12 and secondary luminal confinement 20 occurs through a vascular catheter illustratively inserted in the leg or groin area of the patient. Alternatively, the implantation is by the subclavian artery, axial artery, directly through the wall of the aorta, or another larger artery. The securement 12 has locating features 14 and a stabilization/alignment target 16 that is attached to the securement 12 via a detachable ring 18. The stabilization/alignment target 16 covers an introductory guide channel 22 for passage into secondary luminal confinement 20.

An often-overlooked aspect of cardiac assist devices is the reliable implantation of the same. To this end, an endo-aortic securement 12, a sub-endothelial pocket 20, or a combination thereof are retained in a position within the aorta through resort to an expandable mesh stent S in dilation against the endoluminal wall of the aorta (not shown for visual clarity until FIG. 1M). As a result, the device is positionally stable prior to trans-aortic puncture and during cardiac pumping cycles. It is appreciated that the stent is readily treated with a primary coating to promote long-term stent stability and therefore the device 10 anchored thereto. Such coating substances illustratively include heparin, antibiotics, radiopaque agents, anti-thrombogenic agents, anti-proliferative agents, anti-angiogenic agents; each alone, or in combination. It is further appreciated that a secondary coating overlying the first coating is provided to promote sustained release of the underlying coating substance. Such secondary coatings illustratively include polylactic acid, polyglycolic acid, polyethylene oxide, polycaprolactone, polydioxanones, combinations thereof, and co-polymers thereof.

In certain inventive embodiments, the secondary luminal confinement 20 is formed from a material that induces immune-compatible granulation tissue overgrowth thereon or in-growth therein to effectively render the secondary luminal confinement 20 non-provocative from thrombotic events against the adluminal surface of the secondary luminal confinement 20. Coatings operative herein illustratively include poly-L-lysine (PLL), polylmethyl coguanidine-cellulose sulphate (PMCG)-CS/PLL-sodium alginate (SA), polyethylenimine, poly(dimethyldiallylammonium chloride), chitosan, polyacrylacid, carboxymethylcellulose, cellulose sulfate, pectin, and combinations thereof to form multilayers. It is appreciated that such coatings are readily impregnated with compounds that reduce the immune cascade, these illustratively include heparin and factor H.

FIG. 1B illustrates the introduction of an exo-aortic securement device illustratively including a stapler, which as shown has a circular shape for providing staples 34 (see FIG. 1E) in a circular perimeter, to attach the flange portion of a conduit 24 through the wall W of the vessel V to the securement 12. It is noted that other perimeter shapes illustratively including oval, square, rectangular may be used to secure the flange of the conduit 24 to the securement 12. It is appreciated that other fasteners deployed from a securement device 30 to join the expandable secondary luminal confinement in mechanical communication with a securement within the vessel to a conduit external to the vessel also include tissue adhesives, screws, thread-like sutures, or other mechanical fasteners conventional to surgery. As shown, the exo-aortic securement device 30 (hereinafter referred to as securement device 30) fits around the conduit 24. Upon docking to the securement device 30 to the securement 12, a hemostatic seal is formed in some embodiments. The securement device 30 has complimentary location features 32 to the locating features 14 on securement 12. The conduit 24 has an aperture 26 configured for insertion of an alignment probe 28 that aligns with the stabilization target 16 as shown in FIG. 1C and FIG. 2B, and once aligned the alignment probe 28 penetrates the wall W of the vessel V and stabilizes the stabilization/alignment target 16 as shown in FIG. 1D and FIG. 2C.

In a specific inventive embodiment, the locating features 14 as shown in FIG. 1B are a set of transponders, which may be passive or active, that react to the transmitted seeker signals from the complimentary location features 32 located on the securement device 30 in a similar manner to radio frequency identification RFID based technology. In FIG. 2A the complimentary location features 32 located on the securement device 30 are configured as a transponder/seeker that send signals to the locating features 14 configured as a receiver on the securement 12. Additionally, other locating methods illustratively including light emitting diodes (LED), ultrasound, magnets arrayed as complimentary location features 32, and fluormetry may be used for locating features or fiducial markings for aligning the conduit 24 with the securement 12 to provide an access path into the vessel V.

Once the alignment probe 28 is attached to and stabilizes the stabilization/alignment target 16, an exo-endo aortic securement is established using a series of fasteners illustratively shown as staples 34, where the staples 34 are dispensed from the stapler 30, and the staples 34 pierce through an optional buttress (not depicted), then through the flange portion of the conduit 24 and through the wall W and into the securement 12 that has been implanted in the vessel V, illustratively shown as an aorta in FIG. 1E and FIG. 2D. In FIG. 1F and FIG. 2E a coaxial aortic punch 36 is advanced through the aperture 26 of the conduit 24 to create end-to-side anastomosis of the wall W of the vessel V, and the stabilization/alignment target 16 is detached by breaking the detachable ring 18 that holds the stabilization/alignment target 16 to the securement 12 in FIG. 1G and FIG. 2F. In a specific embodiment a Doppler flow meter or similar flow detection sensor may be associated with the coaxial aortic punch 36, or optionally any of the system components proximate to the anticipated aortic punch site, to check for fluid leaks at the interface of the flange 24, securement 12, and the stent S. It is appreciated that optical coherence tomography (OCT) performs micrometer-scale or catheter-based imaging ultrasound probe, cross-sectional and three-dimensional imaging by measuring the echo time delay of backscattered light in order to preclude an aortic puncture in the vicinity of an aortic wall defect. Optionally the aortic puncture function can be accomplished with a laser source, ultrasonic, water jet, or other conventional techniques to form a geometrically controlled opening in the aortic wall at a defined location. Detachment of the detachable ring 18 may be accomplished by a remote mechanism that illustratively includes electrical detachment or photolabile adhesive. In FIG. 1H and FIG. 2G, the stabilization/alignment target 16 and remnant of the wall W is removed along with the coaxial vascular punch 36 to provide a clear passage between the conduit 24 and the introductory guide channel 22 of the securement 12 that leads into the pocket 20 as shown in FIG. 1I and FIG. 2H. In FIG. 1J a cardiac pumping chamber 38 in a deflated state, optionally delivered in a removable protective cover sheet (not shown), is introduced via the conduit 24 and through the introductory guide channel 22 of the securement 12 and into the secondary luminal confinement 20. In FIG. 1K the cardiac pumping chamber 38 in the deflated state is fully inserted in the pocket 20 with the insertion line 40 is now visible. In a specific embodiment, the cardiac pumping chamber 38 and insertion line 40 are introduced into a patient via an embedded percutaneous access device (PAD) 70 as shown in FIG. 5A. In FIG. 1L and FIG. 2I the cardiac pumping chamber 38 is inflated so as to expand the secondary luminal confinement 20. FIG. 1M is a side cutaway view of the vessel V with a stent S in the region of the securement 12 and the secondary luminal confinement 20, where the pumping chamber 38 is deflated.

FIG. 1N is a side cutaway view of the vessel V with a stent S in the region of the securement 12 and the secondary luminal confinement 20, where the pumping chamber 38 is inflated so as to expand the secondary luminal confinement 20 and move a volume of blood in the vessel V. The inflation cycle of the pumping chamber acts as a cardiac assist device to increase blood ejection from a compromised heart of a patient in need thereof. While the expansion of the pumping chamber 38 is depicted as occluding the aorta, it should be appreciated that this an exaggeration for visual clarity and that such occlusion is implicated in conformational changes in von Willebrand factor commonly associated with clot formation in downstream vasculature.

An embodiment of a system 50 for the attachment and deployment of a cardiac blood pump, or permanent blood pump is described in FIGS. 3A-3I as cross-sectional views and in FIGS. 4A-4C as perspective cross-sectional views of a needle 54 and plunger 64 to assist in the implantation and deployment of a cardiac assist device in accordance with embodiments of the invention. As shown in FIG. 3A-1 and in greater detail in FIG. 3A-2 and FIG. 4A an anvil 52 and needle guide 62 connected to a non-distensible collapsed sub-endothelial pocket 20 (hereinafter referred to as pocket 20) are shown implanted along with a stent S in a patient vessel V illustratively including the aorta. Implantation of the endo-aortic securement anvil 52 and secondary luminal confinement 20 is via conventional methods that illustratively include the use of a vascular catheter. The plunger end 64 of needle 54 is connected to a detachable cable 56, where the cable 56 pulls on the plunger 64 and draws the needle 54 inward into the needle guide 62 where the needle guide 62 directs the needle 54 upward and outward toward the wall W of the vessel V so as to puncture the wall W as shown in FIG. 3B and FIG. 4A. In FIG. 3C a centering probe/telescope 58 is introduced into the patient and is firmly attached to the needle 54. In a specific embodiment the centering probe/telescope 58 is introduced into a patient via a percutaneous access device (PAD) 70 as shown in FIG. 5A. Subsequently, a flanged extra aortic conduit 24 is centered about the centering probe/telescope 58, with fine location placement determined via optional locating features 14 on the anvil 52 and complimentary locating features 32 on the stapler 30 that fits over the conduit 24. In an alternate embodiment, a fluid source for cardiac pump inflation is either a gas or a liquid that are driven periodically into the chamber 38 to create blood movement through a fluid drive system that is wholly implanted and powered by internal batteries or via an external wireless charging device

In a specific inventive embodiment, the locating features 14 as shown above in FIG. 1B may be a set of transponders, which may be passive or active, that react to the transmitted seeker signals from the complimentary location features 32 located on the securement device 30 in a similar manner to radio frequency identification RFID based technology. In a specific embodiment the complimentary location features 32 located on the securement device 30 are configured as a transponder/seeker that send signals to the locating features 14 configured as a receiver on the anvil 52. Additionally, other locating methods illustratively including light emitting diodes (LED), and fluormetry may be used for locating features or fiducial markings for aligning the conduit 24 with the anvil 52 to provide an access path into the vein V. It is appreciated that in some embodiments, an anvil surface has a dimple to deflect a slightly misaligned probe 28 into contact with the pole of a dimple.

In FIG. 3D the stapler 30 is placed about the conduit 24. In the embodiment shown the stapler 30, which as shown has a circular shape for providing staples 34 in a circular perimeter, to attach the flange portion of a conduit 24 through the wall W of the vessel V to the anvil 52. It is noted that other perimeter shapes illustratively including oval, square, rectangular may be used to secure the flange of the conduit 24 to the anvil 52. In FIG. 3E two or more staples 34 are deployed from the stapler 30. In a specific embodiment six staples 34 are deployed around the perimeter of the flange of the conduit 24 and anvil 52. As shown in FIG. 3E counter pressure on the anvil 52 is maintained by pulling up on the centering probe/telescope 58 in an embodiment as the staples 34 are bent upward and back by the anvil 52. In FIG. 3F a vascular wall punch 36 is introduced in the conduit 24 and cuts into and through the wall W. In FIG. 3G and FIG. 4B an electrically detachable ring 66 is exercised to free the needle guide 62. In FIG. 3H the cable 56 is electrically detached from the plunger 64. In FIG. 3I the punched section of the wall W is removed along with the now separated needle guide 62 through the conduit 24 to create a clear channel to the pocket 20. FIG. 4C illustrates the introduction of an operational line 68 through the conduit 24 and into the vessel V.

FIG. 5A is a cross-sectional side view of a percutaneous access device (PAD) 70 implanted in a patient for providing a power or actuating connection 72 via conduit 24 to a ventricular assist device according to an embodiment of the invention. In a specific embodiment the PAD 70 through the skin surface layers (SL) illustratively including the epidermis, dermis, and subcutaneous tissue provides for a semi-permanent connection to an out-of-body power source or pump 78 as shown in FIG. 5D. As is described in greater detail in the prior patents incorporated herein by reference in their entirety, a tube or line 76 can be led from the implanted cardiac pump chamber to a percutaneous access device implanted and projecting through a patient's skin or have wholly implanted fluid drive system and sensor package. Regardless of the nature of the fluid drive system, in some embodiments the fluid includes a marker that when permeating the chamber 38 is indicative of the membrane defining the chamber 38. A marker for a gaseous fluid illustrative includes a diatomic gas that is enriched in either the ortho or para isomers. In a specific embodiment, the diatomic gas is hydrogen that is detected in MRI devices. In still other embodiments, the diatomic gas is isotopically enriched. In instances when the fluid is a liquid, conventional detectable markers are used for detection by techniques illustratively including MRI, ultrasound, and X-ray spectroscopies. It is appreciated that a sensor to detect cardiac pump chamber inflation pressures and/or other operation parameters is readily provided in communication with the fluidics.

The percutaneous access device allows the tube and leads as needed for sensors or other operational aspects, to be operatively connected to or disconnected from an external fluid drive system and controller. In operation, the inflatable cardiac pumping chamber 38 or multiple such chambers are each independently cyclically inflated and deflated with a pressurized fluid with a synchronicity relative to the patient heart. Preferably, the synchronous cyclical inflation and deflation can be based on a set of programmable patient parameters relating to heart function. The fluid driver 78 may supply an inflation fluid as either a gas or a liquid to expand the cardiac pumping chamber 38 within the pocket 20 of the ventricular assist device. It is appreciated that gases other than air are operative with the present invention to induce pump inflation. These gases illustratively include helium, nitrogen, argon, and mixtures thereof. While these gases have lower viscosities than air, such gases necessitate tethering the recipient of an inventive blood pump implant to a compressed gas tank thereby reducing the mobility of the recipient. In a specific embodiment a tracer may optionally be added to the fluid to detect a compromised membrane of the expandable pocket 20. Other fluids such as saline or other hydraulic fluids can serve to actuate the pumping chamber; optionally, a tracer substance such as indocyanine green or fluorescein can be included in the hydraulic liquid for detection of leaks from the pumping chamber.

Optionally, feedback sensors are provided for the operation of an inventive blood pump. Such sensors illustratively include a pressure transducer, an accelerometer, a strain gauge, an electrode, and species-specific sensors such as pH, oxygen, creatine, nitric oxide or MEMS versions thereof. The output of such a sensor being transmitted as an electrical or optical signal to monitoring and regulatory equipment exterior to the body of the recipient.

Embodiments of the inventive cardiac pump alone or a plurality of such pumps in the aggregate displaces from about 20 to 70 cubic centimeters of blood upon inflation; each alone or collectively when several chambers are implanted and operating collectively. In a particular inventive embodiment, 50 to 70 cubic centimeters of blood are displaced per heartbeat by the present invention so as to allow an individual having an inventive pump implanted an active lifestyle. In still other embodiments, 60 to 65 cubic centimeters of blood per patient heartbeat by the present invention. The long axis of the pocket and the pumping chamber are aligned along the long axis of the aorta. Alternatively, the pumping chamber is symmetric in at least two orthogonal axes, or the pumping chamber long axis extends helically, or in some other non-linear form in a local segment of the aorta.

FIG. 5B illustrates the use of an implanted transcutaneous energy transfer module (TET) for providing one or more power or actuating connections (72, 72′) via conduits 24 that are connected to one or more cardiac assist devices according to an embodiment of the invention. In a specific embodiment two or more ventricular assist devices may be placed in an aorta of a patient.

FIG. 5C illustrates multiple power or actuating connections 72 emanating from a single PAD 70 that is connected to an external power supply/pump 78 via external line 76.

FIG. 5E illustrates multiple cardiac assist devices along the aorta of a patient. It is appreciated that multiple cardiac pump chambers are synchronized together to blood flow in the aorta, with each of the multiple devices have an independent fluid source and drive system, else two or more cardiac pump chambers are manifolded to share a single fluid source and/or drive system

FIGS. 6A-6F are a series of cross-sectional side views showing the implementation and actuation of a cardiac assist device in accordance with embodiments of the invention. In FIG. 6A an initial stent S and pocket 20 with a securement 12 are placed in the vessel V of the patient. Optical coherence tomography (OCT) is a recently developed technology that uses infrared light to generate micrometer-scale cross-sectional images (Science. 1991; 254:1178-1181), OCT is optionally used in the present invention to assess the microstructure of the aortic wall in the intended region of device placement to avoid fixturing of a device proximal to an aortic wall defect. Typically, OCT resolutions of 4 to 16 are adequate to assess aortic wall integrity. OCT is readily performed using a conventional intravascular OCT endoscope. It is appreciated that OCT with a micro-motor catheter affords high frame per second imaging, while MEMS-tunable vertical cavity surface emitting laser (VCSEL) OCT has still other advantages in terms of miniaturization and imaging quality (T-H Tsai et al, “Ultrahigh speed endoscopic optical coherence tomography using micromotor imaging catheter and VCSEL technology” Biomed Opt Express. 2013 Jul. 1; 4(7): 1119-1132.). OCT is also readily combined with fluorescent contrast for intravascular atherosclerotic imaging or embolism imaging.

In FIG. 6B a conduit 24 is secured to the securement 12 and the wall W of the vessel V. In FIG. 6C a clear channel is created between the conduit and the pocket 20. In FIG. 6D the pumping chamber 38 is introduced into the expandable pocket 20. FIG. 6E illustrates the state of the vessel V when the pumping chamber 38 is deflated, and FIG. 6F shows the state of the vessel with volume displacement with the pumping chamber 38 inflated in the pocket 20.

In some inventive embodiments a vacuum source is applied to the pumping chamber 38 or the interstitial space between the pocket 20 and the pumping chamber 38. Periodic vacuum application is readily applied for an extended period of time with limited or no inflation or as part of a pump inflation cycle. Vacuum application is used for various functions illustratively including micro-leak detection in the pocket 20 or the pumping chamber 38, as well as promoting evaporation of condensate.

FIGS. 7A-7D illustrate an endovascular procedure where the components for an aortic assist device may be delivered in two stages with the elimination of the stage that introduces the secondary luminal confinement (expandable pocket) 20. It is appreciated that the elimination of the secondary luminal confinement leads to a less invasive procedure for both the initial implantation, and for the potential future replacement of the aortic assist device. It is also appreciated that a stent may also be introduced at the insertion site of aortic assist device 80, but is left out for clarity in the drawings.

In the first stage, the securement 12 is delivered into the vein (V), for example by a groin catheter The second stage introduces the aortic assist device 80 with a flexible encasement 82 and a balloon 84 inside, and may enter the vein via the extra aortic conduit 24 after the flange of the conduit 24 is mounted to the securement 12 to deliver the aortic assist device 80 through the conduit 24. FIG. 7A illustrate the joining of the securement 12 to the flange of the conduit 24 via methods as described in the embodiments above to form an access channel that includes aperture 26 and introductory guide channel 22. In FIG. 7B the delivery of the flexible encasement 80 begins through the access channel. In FIG. 7C the aortic assist device 80 in a deflated state is fully inserted in the vein with the insertion line 40 that is now visible. In a specific embodiment, the flexible encasement 80 and insertion line 40 are introduced into a patient via an embedded percutaneous access device (PAD) 70 as shown in FIG. 5A. FIG. 7D illustrates the inflation of the aortic assist device 80 via inflation of the balloon 84.

FIGS. 8A and 8B are cross-sectional views of the aortic assist device 80 with a flexible encasement 82 and the balloon 84 inside (shown in dotted lines). The flexible encasement 82 protects the balloon from the blood flow in the vein, and guards against a potential failure of the balloon 84. In FIG. 8A the balloon is in a deflated state, and in FIG. 8B the balloon is inflated.

It is appreciated that the flexible encasement 82 may be readily treated with a primary coating. Examples of coating substances illustratively include heparin, antibiotics, radiopaque agents, anti-thrombogenic agents, anti-proliferative agents, anti-angiogenic agents; each alone, or in combination. It is further appreciated that a secondary coating overlying the first coating is provided to promote sustained release of the underlying coating substance. Examples of secondary coatings illustratively include polylactic acid, polyglycolic acid, polyethylene oxide, polycaprolactone, polydioxanones, combinations thereof, and co-polymers thereof.

In certain inventive embodiments, the flexible encasement 82 may be formed from a material that induces immunocompatible granulation tissue overgrowth thereon or in-growth therein to effectively render the secondary luminal confinement 20 non-provocative from thrombotic events against the adluminal surface of the flexible encasement 82. Coatings operative herein illustratively include poly-L-lysine (PLL), polylmethyl coguanidine-cellulose sulphate (PMCG)-CS/PLL-sodium alginate (SA), polyethylenimine, poly(dimethyldiallylammonium chloride), chitosan, polyacrylacid, carboxymethylcellulose, cellulose sulfate, pectin, and combinations thereof to form multilayers. It is appreciated that such coatings are readily impregnated with compounds that reduce the immune cascade, these illustratively include heparin and factor H.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

1. A cardiac assist device comprising:
an inflatable cardiac pumping chamber with an integrally textured polymeric membrane contacting blood upon insertion in a subject aorta;
a drive line in fluid communication with said an inflatable cardiac pumping chamber; and
an external drive unit or fluid supply in fluid communication with said drive line.

2. The cardiac assist device of claim 1 wherein said integrally textured polymeric membrane is polyurethane.

3. The cardiac assist device of claim 1 wherein said integrally textured polymeric membrane has at least one pleat formed therein.

4. The cardiac assist device of claim 3 wherein said at least one pleat is a plurality of pleats.

5. The cardiac assist device of claim 4 wherein said plurality of pleats extends substantially along a long axis length of said integrally textured polymeric membrane.

6. The cardiac assist device of claim 4 wherein said plurality of pleats are substantially parallel.

7. The cardiac assist device of claim 3 wherein said at least one pleat forms a spiral.

8. The cardiac assist device of claim 7 further comprising a plurality of substantially parallel pleats.

9. The cardiac assist device of claim 8 wherein said plurality of substantially parallel pleats intersects said spiral.

10. The cardiac assist device of claim 1 wherein said inflatable cardiac pumping chamber is sutured to the aorta.

11. The cardiac assist device of claim 1 wherein said inflatable cardiac pumping chamber is at least one balloon inserted within the aorta.

12. The cardiac assist device of claim 1 wherein said external drive unit further comprises a pump modifying a pressure of fluid in said inflatable cardiac pumping chamber with a periodicity to aid in blood movement through the aorta.

13. (canceled)

14. The cardiac assist device of claim 1 further comprising a percutaneous access device intermediate between said drive line and said external drive unit or said fluid supply.

15. The cardiac assist device of claim 1 further comprising an immuno-isolation coating on said integrally textured polymeric membrane.

16. The cardiac assist device of claim 15 wherein said immuno-isolation coating is poly-L-lysine (PLL), polylmethyl coguanidine-cellulose sulphate (PMCG)-CS/PLL-sodium alginate (SA), polyethylenimine, poly(dimethyldiallylammonium chloride), chitosan, polyacrylacid, carboxymethylcellulose, cellulose sulfate, pectin, or combinations thereof.

17. The cardiac assist device of claim 16 wherein said immuno-isolation coating further comprises a compound that reduces the immune cascade.

18. The cardiac assist device of claim 17 wherein said compound is heparin or factor H.

19. An improved inflatable cardiac pumping chamber having a membrane moving to change a volume of the chamber based on fluid input from an inflation source, a drive line in fluid communication with the inflatable cardiac pumping chamber and the inflation source, wherein the improvement lies in: the membrane being an integrally textured polymeric membrane contacting blood upon insertion in a subject aorta.

20. The improved inflatable cardiac pumping chamber of claim 19 wherein the improvement further lies in: said integrally textured polymeric membrane being polyurethane.

21-27. (canceled)
